(19) United States
(12) Patent Application Publication
(10) Pub. No.: US 2022/0296742 A1
(43) Pub. Date: Sep. 22, 2022

(54) ANTIBACTERIAL INTERIOR COMPONENTS AND METHODS FOR USE THEREOF

(71) Applicant: GM GLOBAL TECHNOLOGY OPERATIONS LLC, Detroit, MI (US)

(72) Inventors: Nancy L. JOHNSON, Northville, MI (US); Janet C. ROBINCHECK, Sterling Heights, MI (US); Judith H. CLARK, Hartland, MI (US); Adam F. GROSS, Santa Monica, CA (US); Shanying CUI, Calabasas, CA (US); Russell MOTT, Malibu, CA (US)

(21) Appl. No.: 17/205,955

(22) Filed: Mar. 18, 2021

(51) Int. Cl. A61L 2/08; B60S 1/64

(52) U.S. Cl. CPC A61L 2/084; B60S 1/64; A61L 2202/11; A61L 2202/14

(57) ABSTRACT

An antibacterial interior component, such as a door handle component, is provided in a vehicle that includes at least one touch surface in a confined space having a shadowed region. A light source includes a light emitting diode (LED) that generates light having a wavelength of ≥about 375 nm to ≤about 425 nm directed towards the at least one touch surface for killing bacteria. A thermally conductive component in heat transfer relationship with the light source to transfer heat to a heat sink either in or adjacent to the antibacterial interior component. Methods of operating the self-sanitizing antibacterial interior component are also provided.

### INTRODUCTION

This section provides background information related to the present disclosure which is not necessarily prior art.

The present disclosure pertains to antibacterial interior components for vehicles and methods for use thereof.

Transmission of diseases and odors in vehicles are a major concern, especially for ride-sharing or multi-occupant vehicles. Certain interior components of vehicles have buttons, switches, levers, or other surfaces that are frequently touched by users (“high interaction vehicle surfaces” or “high touch areas”). Thus, cleaning of these high touch surfaces to remove pathogenic bacteria or unwanted odors generated by bacteria in human sweat and food residue is desirable. While various disinfecting strategies have been employed in vehicles, many of these rely on physical cleaning by a human, such as applying a cleanser directly to the surface, sometimes followed by physical contact, like wiping.

However, certain high touch regions of interior components may be difficult to clean as they are disposed in a confined area lacking easy access or disposed in shadowed regions that are not penetrated by external incident light. For example, the interior portions of handles, latches, cup holders, center console compartments, and the like, may have high touch surfaces that cannot be easily accessed for cleaning. Thus, it would be desirable to target treatment on high interaction vehicle surfaces so that bacteria transmittance can be reduced in difficult to access regions of interior components. Thus, there remains a need for self-cleaning and self-sanitizing surfaces in an interior component capable of safely minimizing bacteria and other contaminants in regions of the interior component that are difficult to access.

### SUMMARY

This section provides a general summary of the disclosure, and is not a comprehensive disclosure of its full scope or all of its features.

The present disclosure relates to an antibacterial interior component capable of self-cleaning disposed in a vehicle. The antibacterial interior component has at least one touch surface in a confined space having a shadowed region and a light source including a light emitting diode (LED) that generates light having a wavelength of greater than or equal to about 375 nm to less than or equal to about 425 nm directed towards the at least one touch surface for killing bacteria. The antibacterial interior component also has a thermally conductive component in heat transfer relationship with the light source to transfer heat to a heat sink either in or adjacent to the antibacterial interior component.

In certain aspects, the light source includes a light emitting diode (LED) having an optical power output capacity of greater than or equal to about 1 W.

In certain aspects, the light source is configured to have a first operational mode for killing bacteria on the at least one touch surface having an optical power output of greater than or equal to about 0.5 W and a second operational mode for illumination having an optical power output of less than or equal to about 0.5 W.

In certain aspects, the first operational mode is configured for killing greater than or equal to about 90% of bacteria initially present on the at least one touch surface.

In certain aspects, the light source generates a radiant fluence on the at least one touch surface of greater than or equal to about 5 J/cm² to less than or equal to about 50 J/cm².

In certain aspects, an irradiance on the at least one touch surface is greater than or equal to about 1 mW/cm².

In certain aspects, the antibacterial interior component is selected from the group consisting of: a door handle, a cup holder, a glove compartment, a latch, a handle, a steering wheel, and combinations thereof.

In certain aspects, a region surrounding the light source does not exceed a temperature of greater than about 100° C.

In certain aspects, the thermally conductive component has a thermal conductivity of greater than or equal to about 20 Wm/K.

In certain aspects, the thermally conductive component is a thermal heat bridge attached to the light source and to a solid heat sink.

In certain aspects, the antibacterial interior component is a door handle, the light source is disposed in or near a bezel that surrounds the door handle, and the heat sink is a door panel.

In certain aspects, the thermally conductive component is a thermal bridge connecting the light source to a solid component in a door panel or the thermally conductive component includes a plurality of heat exchange fins.

In certain aspects, the thermally conductive component includes a phase change material that is configured to undergo an endothermic reaction to absorb heat.

In certain aspects, the thermally conductive component is a potting compound or a heat spreader disposed on a surface of the antibacterial interior component.

In certain aspects, the antibacterial interior component is a door handle and the light source is disposed in the door handle.

The present disclosure also relates to an antibacterial door handle component in a vehicle that may include at least one touch surface on a door handle in a door handle component in a door panel. The door handle component includes a confined space having a shadowed region. The antibacterial door handle component also includes a light source including a light emitting diode (LED) associated with the door handle component that generates light having a wavelength of greater than or equal to about 375 nm to less than or equal to about 425 nm directed towards the at least one touch surface on the door handle for killing greater than or equal to about 90% of bacteria present on the at least one touch surface. A thermally conductive component in heat transfer relationship with the light source is also included to transfer any heat generated to the door panel.

The present disclosure further relates to a method of operating an antibacterial interior component of a vehicle. The method includes activating a light source including a light emitting diode (LED) in a first operational mode. In the first operational mode, the LED generates blue light having a wavelength of greater than or equal to about 375 nm to less than or equal to about 425 nm, which is directed towards at least one touch surface in a confined space of the antibacterial interior component having a shadowed region. In this manner, greater than or equal to about 90% of bacteria initially present on the at least one touch surface is killed. The method also includes transferring heat from the light source to a thermally conductive component either in or adjacent to the antibacterial interior component, so that a temperature in a region within or adjacent to the antibacterial interior component is less than or equal to about 100° C. during the activating.

In certain aspects, the method further includes activating the light source including the light emitting diode (LED) in a second operational mode distinct from the first operational mode to illuminate the antibacterial interior component having a shadowed region. The first operational mode for killing bacteria has an optical power output of greater than or equal to about 0.5 W, while the second operational mode for illumination has an optical power output of less than or equal to about 0.5 W.

In certain aspects, an irradiance on the at least one touch surface during the first operational mode is greater than or equal to about 10 mW/cm².

In certain aspects, the light source generates a radiant fluence on the at least one touch surface during the first operational mode of greater than or equal to about 5 J/cm² to less than or equal to about 50 J/cm².

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 shows a vehicle door having an antibacterial door handle component disposed in a bezel having a high energy light source in accordance with certain aspects of the present disclosure.

FIG. 2 shows an interior side of a door handle component like that in FIG. 1.

FIG. 3 shows a vehicle door having an antibacterial door handle component disposed in a bezel having a high energy light source connected to a thermally conductive component in the form of a thermal bridge to transfer heat to a heat sink in accordance with certain aspects of the present disclosure.

FIG. 4 shows another view of the antibacterial door handle component in FIG. 3 where the high energy light source is activated in an antibacterial cleaning mode in accordance with certain aspects of the present disclosure.

FIG. 5 shows an exploded view of an antibacterial door handle component disposed in a bezel having a high energy light source connected to a thermally conductive component in the form of a thermal bridge to transfer heat to a heat sink in the door panel behind the antibacterial door handle component in accordance with certain aspects of the present disclosure.

FIG. 6 shows an antibacterial door handle component disposed in a bezel having a high energy light source connected to a thermally conductive component in the form of heat exchange fins to transfer heat in accordance with certain aspects of the present disclosure.

FIG. 7 shows a vehicle door having an antibacterial door handle component having an internal high energy light source in accordance with certain aspects of the present disclosure.

FIG. 8 shows a front view of the antibacterial door handle component in FIG. 7 having the internal high energy light source.

FIG. 9 shows a back view of the antibacterial door handle component in FIG. 7 having the internal high energy light source.

FIG. 10 is a flow chart representing methods of operating an antibacterial interior component in a vehicle in accordance with certain aspects of the present disclosure.

FIG. 11 is a schematic of a vehicle interior in which self-sanitizing antibacterial interior components may be incorporated in accordance with certain aspects of the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

Example embodiments will now be described more fully with reference to the accompanying drawings.

In various aspects, the present disclosure provides an antibacterial or self-sanitizing interior component in a vehicle and methods for killing bacteria in such an interior component. Generally, the interior component according to the present disclosure may have at least one touch surface in a confined space having a shadowed region. The high touch surface region may be formed of a material such as metal, polymeric material (e.g., thermoplastic olefin (TPO), polypropylene, leather, vinyl), leather, cloth, fabric (e.g., woven foam-backed fabric), or any other suitable material typically used for forming interior components in a vehicle. A confined space in an interior component is an area of a passenger compartment that is a relatively small volume, for example, less than or equal to about 2 cubic feet or optionally less than or equal to about 1 cubic foot, that is at least partially shadowed. By shadowed, it is meant that at least a portion of the interior component has surfaces that are not visible to an occupant of a vehicle, so that minimal incident light reaches the shadowed region.

The interior component includes a light source comprising a light emitting diode (LED) that generates blue light having a wavelength of greater than or equal to about 375 nm to less than or equal to about 425 nm. Blue light in this range of wavelengths can have an antibacterial effect that can kill both pathogenic and odor causing bacteria without damaging surfaces (e.g., destabilizing or degrading interior component surfaces) or endangering humans (in contrast to ultraviolet light, such as UV-C light, for example). Porphyrin molecules in bacteria involved with their metabolism absorb blue light, are damaged, and cause the bacteria to generate reactive oxygen species that further damage the bacteria. The blue light also is absorbed by bacterial membrane proteins, denatures the proteins, and breaks open cell walls. The end effect is the death of bacteria. In certain variations, the blue light has a wavelength of greater than or equal to about 385 nm to less than or equal to about 410 nm, for example, about 390 nm or 405 nm. In certain variations, the blue light is greater than or equal to about 385 nm to less than or equal to about 395 nm, for example, 390 nm. As such, the light source generates and emits blue light that is directed toward the shadowed region of the at least one touch surface for killing bacteria.

The light source may comprise one or more LEDs capable of generating such blue light. One or more LEDs may be a high power LED that in certain operational modes generates an output that achieves a bactericidal effect. Such a high power LED may emit a total of greater than or equal to about 0.5 W of optical power at its surface (a “0.5 W LED”), optionally greater than or equal to about 1 W of optical power at its surface (a “1 W LED”), optionally greater than or equal to about 2 W of optical power (a “2 W LED”), optionally greater than or equal to about 3 W of optical power (a “3 W LED”), optionally greater than or equal to about 4 W of optical power (a “4 W LED”), and in certain variations, optionally greater than or equal to about 5 W optical power at its surface (e.g., a “5 W LED”). Notably, optical power differs from electrical power. Thus, a high optical power 5 W LED has a greater electrical power rating than a 5 electrical watt LED, because LEDs are not 100% efficient from power source optical power and the optical power is usually only about ⅓ of the power rating (e.g., ⅓ of 5 W). For example, 90 W of optical power in an LED would translate to 270 W of electrical power. Thus, a high optical power 5 W LED may be a 15 W or greater electrical watt LED. In certain aspects, two or more high optical power (e.g., high power 5 W LEDs) can be used to illuminate the shadowed regions of the confined space in the interior component. Moreover, multiple high power LEDs may be used to generate a desired level of flux or power on target areas suitable for providing a predetermined bactericidal effect. In certain variations, the light source consists of only high power LEDs generating blue light. In terms of optical power, it may be expressed in one aspect as radiant flux (W) that characterizes a total radiance energy per unit time emitted from the LED power source. Irradiance or flux density (W/cm²) is radiant flux received by a surface per surface area. Radiant fluence or radiant exposure or radiant energy (J/cm²) is cumulative radiant energy received by surface per unit area over a given period of time.

In certain aspects, where the light source may be configured to have a first operational mode and a distinct second operational mode. The integrated lighting thus has at least two intensity levels to provide night time illumination, as well as bacterial kill. The first operational mode is for killing bacteria on the at least one touch surface and has an optical power output or radiant flux of greater than or equal to about 0.5 W, optionally greater than or equal to about 1 W, optionally greater than or equal to about 2 W, optionally greater than or equal to about 3 W, optionally greater than or equal to about 4 W, and in certain variations, optionally greater than or equal to about 5 W. A second operational mode that is used merely for illumination, for example, when the vehicle is occupied and/or during vehicle operation, may have an optical power output of less than or equal to about 0.5 W. The first operational mode is configured for substantially killing bacteria, for example, by killing greater than or equal to about 90% of bacteria initially present on the at least one touch surface, optionally greater than or equal to about 95% of bacteria, optionally greater than or equal to about 97% of bacteria, optionally greater than or equal to about 98% of bacteria, and in certain variations, optionally greater than or equal to about 99% of bacteria initially present on the at least one touch surface. By way of example, blue light (375-425 nm) can reduce bacteria populations on automotive surfaces by up to 99.5% without causing any surface damage.

Two 5 W LEDs can generate an irradiance or flux density of greater than or equal to about 0.05 to less than or equal to about 0.35 W/cm², optionally greater 0.2 to less than or equal to about 0.35 W/cm² on a target touch surface of the interior component. In certain variations, irradiance or flux density of blue light over a given target surface area to be treated reaches a desired cumulative irradiance or radiant fluence. In certain aspects, an irradiance or flux density on the high touch target surface region to be treated to kill bacteria in other words, to achieve a bactericidal effect, may be greater than or equal to about 1 mW/cm², optionally greater than or equal to about 5 mW/cm², optionally greater than or equal to about 10 mW/cm², optionally greater than or equal to about 20 mW/cm², optionally greater than or equal to about 30 mW/cm², optionally greater than or equal to about 40 mW/cm², and optionally greater than or equal to about 50 mW/cm². The localized flux in a given region may be higher. In certain aspects, the irradiance may be greater than or equal to about 5 mW/cm² to less than or equal 90 mW/cm² in a target surface region, generally corresponding to a shadowed region of an interior component.

In certain aspects, the high energy light source generates a cumulative energy or radiant fluence on the at least one touch surface of greater than or equal to about 5 J/cm² to less than or equal to about 50 J/cm² to kill a desired amount of bacteria, for example, at the levels previously described above. In certain variations, the radiant fluence on the at least one touch surface of greater than or equal to about 15 J/cm² to less than or equal to about 25 J/cm², for example, optionally about 20 J/cm².

Low power lighting components have typically been incorporated into main areas or open regions of the passenger component. Light from typical illumination sources in a passenger compartment of a vehicle, for example, in a headliner, in a dashboard, or on trim components, only illuminates line-of-sight regions within the interior components. This leaves interior components with confined spaces and/or shadowed regions hidden from exposure by light. However, many of these confined spaces having at least one shadowed region are high touch surfaces that require regular cleaning. Thus, incorporating blue light into typical locations in a vehicle would not penetrate the high touch surfaces in shadowed regions of the interior components.

As noted above, these shadowed regions are also often challenging to physically clean by wiping or applying cleanser, as they may be obscured or relatively inaccessible. For example, this may include the back of door handles disposed in a bezel, cup holders, and the like. Furthermore, even where blue light may reach high touch surfaces, full cabin illumination can require at least one hour of treatment time with blue light as compared to locally illuminating target regions of select components for short (e.g., less than or equal to about 20 minute treatments), which requires substantially less power. Integrating LEDs directly into interior components having high touch regions in accordance with certain aspects of the present disclosure has been found to enhance flux of blue light, which results in faster treatment times and lower overall electrical power requirements.

The system may include a processor in the vehicle that is in communication with the high energy light source. The processor may be in communication (e.g., hard wired or wireless) with the high energy light source by any suitable manner and is configured to enable/disable the high energy light source in the cabin or occupant zone of the vehicle. As will be discussed in greater detail below, the processor may include at least one algorithm in processing steps to enable or disable the high energy light source in the cabin.

The system may also include one or more sensors in communication with the vehicle processor for providing input as to whether it is dark on the exterior of the vehicle and/or whether the cabin has a passenger/occupant or is occupied. The system may include a plurality of sensors disposed on the exterior of the vehicle or within the cabin or occupant zone of the vehicle. In certain variations, the sensor(s) are in communication (e.g., hard wired or wireless) with the processor by any suitable conventional manner to provide data input to the processor related to occupancy of the vehicle. Such input to the processor is used by the processor to activate or deactivate the high energy light source.

The sensor may be any suitable sensor that may provide such input to the processor to activate or deactivate the high energy light source in the interior component within the vehicle. For example, the sensor(s) may include a light sensor, a motion sensor, an optical sensor, a mass sensor, a pressure sensor, a temperature sensor, an ultrasonic sensor, or an infrared sensor, or any other known sensor.

Thus, the present disclosure contemplates using a high power LED to provide an antibacterial interior component of a vehicle. This lighting can sanitize normally shadowed surfaces that are hard to thoroughly sanitize with chemicals, UV light, or more centralized interior lighting systems. The antibacterial interior components described herein automatically sanitize without using chemicals, requiring human action, and further will not damage surfaces (like UV light does). However, one or more of such high power LEDs generate significant heat. However, when the high power LEDs are used near or in confined spaces of interior components, local heating may become excessive. Such regions often do not have adequate fluid flow rates (e.g., circulating air or coolants) to remove heat and cool the high power LEDs. Thus, many interior components do not have airflow on the back surfaces for cooling. In certain variations, the antibacterial interior components further comprise directing heat to a solid in the component body or to an adjacent structural element. This advantageously results in heat sinking to the component itself or to an adjacent or nearby structural member, because static air behind a component will otherwise typically not remove heat from a LED or a conventional heat sink.

The antibacterial interior components of the present disclosure include at least one thermally conductive component in heat transfer relationship with the light source to transfer heat. The at least one thermally conductive component may also be in heat transfer relationship to a solid material that serves as a heat sink, which is either in or adjacent to the antibacterial interior component. In this manner, the antibacterial interior components in the region surrounding the light source do not exceed a temperature of greater than about 100° C., preventing damage to the structures and materials associated with the LEDs and the confined space of the component.

The antibacterial interior components may be disposed in an interior portion of a vehicle within an occupant zone. The antibacterial interior components may be any components touched by or exposed to an occupant (e.g., driver, passenger) that are cast at least partially in shadow and may define a confined space as described above. It is to be understood that the antibacterial interior components may include any of those with high touch surfaces or those that may accumulate bacteria and/or soil, such as handles, including door handles, locks, latches, switches, buttons, displays, steering wheels, lift gates, cup holders, consoles, such as center console storage, mobile device docks and charging stations, electrical outlets, including USB ports, glove boxes, or any other suitable component in the occupant zone of the vehicle without departing from the spirit of the present disclosure.

According to one embodiment of the present disclosure, FIG. 11 shows a vehicle 350 including a chassis 352 and a body 354 supported by the chassis 352. As shown, the body 354 includes a motor compartment 356 and a cabin or interior 358 that one or more occupants (e.g., driver or passengers) can occupy. The vehicle 350 further includes at least one self-sanitizing antibacterial component for bacteria irradiation from the cabin 358. While not shown, it will be appreciated that the self-sanitizing antibacterial component may also be on an exterior of the vehicle 350, as well. In certain variations, the antibacterial interior component is selected from the group consisting of: a door handle 360 in a door 362, a cup holder 370, a glove compartment 372, a latch 374, a center console 380, a steering wheel 382, and the like, and combinations thereof.

It should be noted that the antibacterial components provided by the present technology are particularly suitable for use in an automobile or in other vehicles (e.g., motorcycles, boats, tractors, buses, motorcycles, trains, mobile homes, campers, and tanks), in alternative aspects, they may also be used in a variety of other industries and applications, including aerospace components, consumer goods, devices, buildings (e.g., houses, offices, sheds, warehouses), office equipment and furniture, and industrial equipment machinery, agricultural or farm equipment, or heavy machinery, by way of non-limiting example.

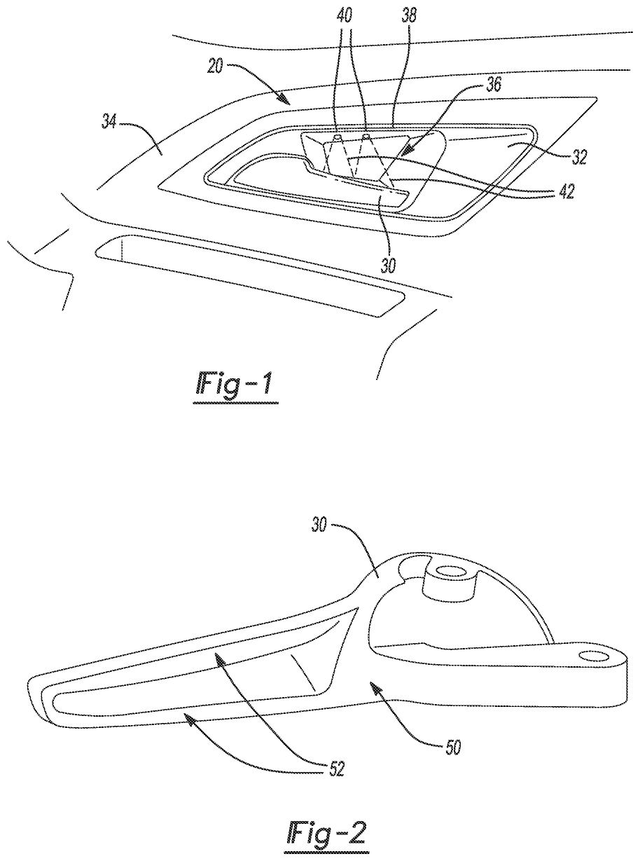

FIGS. 1 and 2 show one non-limiting example of an antibacterial interior component 20 in the form of a door handle 30 disposed in a bezel 32 that is integrated into a door (an interior door panel 34) of a vehicle. The bezel 32 defines a confined space that includes a shadowed area or region 36 beneath an upper portion 38. A plurality of high power LED light sources 40 are integrated into the bezel 32. The LED light sources may be mounted flush with any surface of the interior component and may be disposed behind a transmissive or transparent lens or cover. When activated, the LED light sources 40 generate blue light 42 that is directed towards handle 30, as described above. FIG. 2 shows an interior or back side 50 of handle 30 that faces towards the bezel 32 in FIG. 1. The handle 30 includes high touch surface regions 52 that are contacted by an occupant as they enter or exit the vehicle when the door is opened by the handle 30. As shown in FIG. 1, these high touch surface regions 52 on the back side 50 are cast in a shadow in shadowed region 36 and are not illuminated. Further, these high touch surface regions 52 are not readily accessible for thorough cleaning by applying a cleanser or wiping. Thus, when the blue light 42 is generated by the high power LED light sources 40 of the antibacterial interior component 20, these high touch surface regions 52 can be treated to substantially kill bacteria.

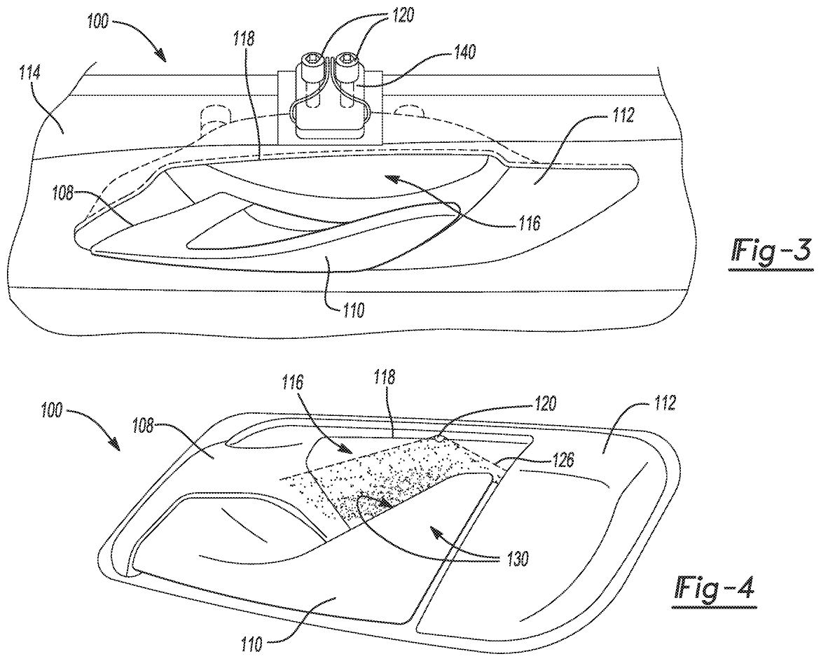

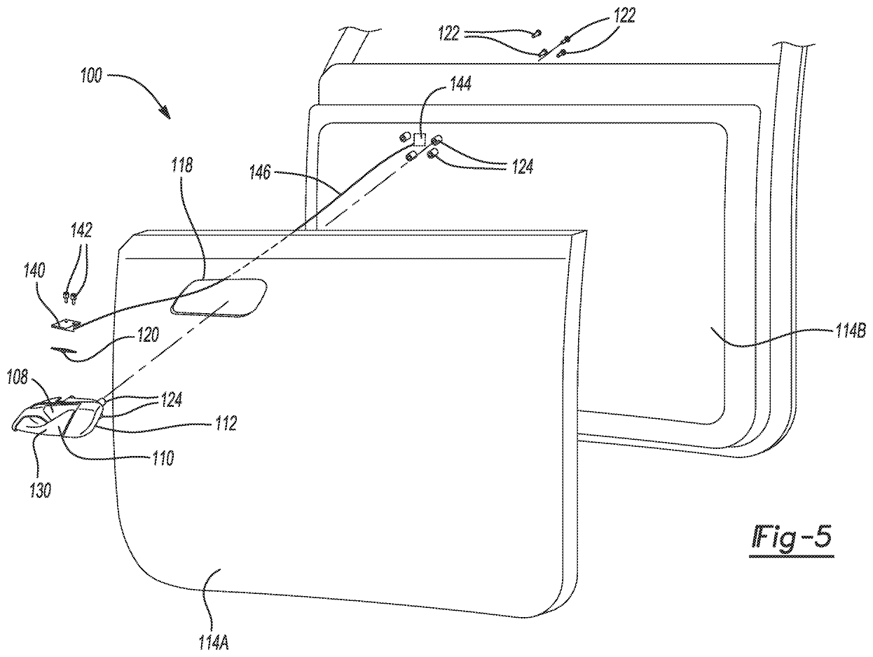

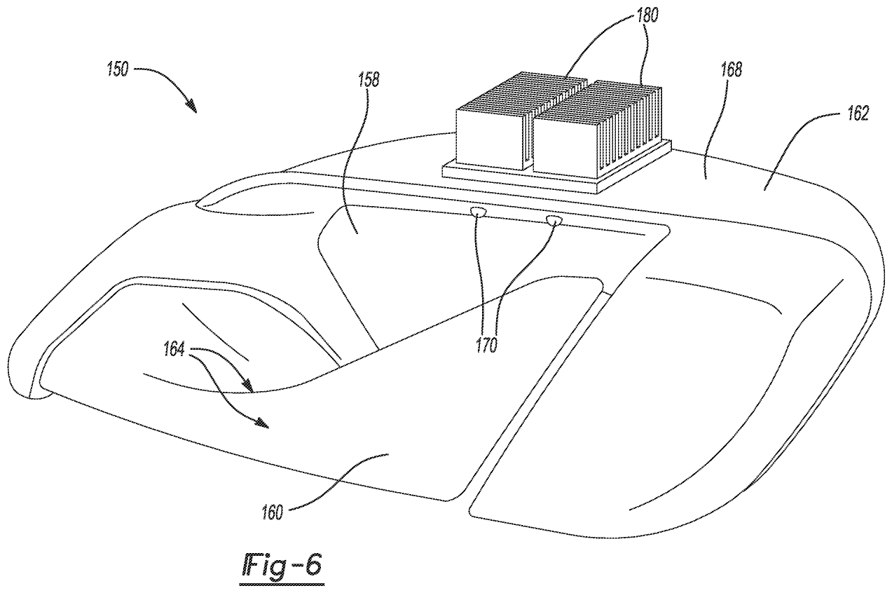

FIGS. 3-5 show another antibacterial interior component 100 includes a door handle component 108 that includes a door handle 110 disposed in a bezel 112 that is integrated into a door panel of a vehicle. Notably, the door panel may be a multicomponent panel assembly, for example, including a first interior door panel component 114A and a second door panel component 114B, but may differ from the variations shown in FIG. 5, which is merely one example for purposes of illustration. The door handle component 108 is attached to the first door panel 114A by a plurality of fasteners 122 are seated in cooperating mounting components 124 respectively in the door handle component 108 and the first and second door panel components 114A and 114B. The bezel 112 is disposed and fixed between the door handle component 108 and the first door panel 114. The handle 110 includes high touch surface regions 130 that are contacted by an occupant as they enter or exit the vehicle when the door is opened by the door handle 110. The bezel 112 may seat around the door handle component 108. The bezel 112 may define a roof or upper portion 118 (or the upper portion 118 may be defined by the region of the first door panel component 114A receiving the bezel 112). Together, the door handle component 108 and the upper portion 118 of the bezel 112 or first door panel component 114A define a shadowed region 116.

One or more high power LED light sources 120 are disposed and optionally attached to the bezel 112 and/or door handle component 108. As will be appreciated by those of skill in the art, various other conventional components and connectors may be present that are not described and shown in FIGS. 3-5 for simplicity. When activated, as shown in FIG. 4, the LED light sources 120 generate blue light 126 that is directed towards at least a portion of the high touch regions 130 on the door handle 110.

The antibacterial interior component 100 also includes at least one thermally conductive component in the form of a thermal bridge 140 in heat transfer relationship with the LED light source 120 to transfer heat. The thermal bridge 140 may be in heat transfer relationship to a solid material that serves as a heat sink, which is the solid door panel (first door panel component 114A or second door panel component 114B) in FIG. 5. As shown in FIG. 5, the thermal bridge 140 is connected to the first door panel component 114A and in designs requiring substantial heat transfer, may also be connected to a thermal interface 144 on the second door panel component 114B. The thermal interface 144 is an area where heat is transferred to second door panel component 114B. The thermal interface 144 may be a direct connection where two materials contact or touch, it can have a thermal interface material disposed on it, such as thermal grease or materials discussed previously above, or it can be physically connected via a thermally conductive conduit, for example, soldered. As shown, a second thermal conductor 146 is physically connected to the thermal interface 144, but as will be appreciated by those of skill in the art, the thermal interface 144 and the second thermal conductor 146 to the second door panel component 114B are merely optional.

The LED light source 120 may be physically attached to the thermal bridge 140 or may have a thermal interface material disposed between these components. In certain variations, the thermal bridge 140 may be soldered to the LED light source 120. The thermally conductive component, such as thermal bridge 140 and/or optional thermal interface 144 may be formed of a material that has a thermal conductivity (K) of greater than or equal to about 20 Wm/K at standard temperature and pressure conditions, optionally greater than or equal to about 30 Wm/K, optionally greater than or equal to about 40 Wm/K, optionally greater than or equal to about 50 Wm/K, optionally greater than or equal to about 100 Wm/K, optionally greater than or equal to about 150 Wm/K, optionally greater than or equal to about 200 Wm/K, optionally greater than or equal to about 250 Wm/K, optionally greater than or equal to about 300 Wm/K, and in certain variations, optionally greater than or equal to about 350 Wm/K. The thermally conductive component or interface may be formed of a material that has a similar thermal conductivity to the surrounding materials (e.g., components in the door panel) that serve as a heat sink. In certain aspects, the thermally conductive component, like thermal bridge 140 or optional thermal interface 144, may be formed of a thermally conductive metal, such as copper, silver, gold, zinc, tungsten, aluminum, steel, or alloys or compounds thereof, including aluminum nitride. Other suitable thermally conductive materials include graphite, graphene, silicon carbide, and the like. The solid heat sink attached to the thermal bridge may have a high heat capacity to absorb heat transferred from the light sources. By way of example, a suitable specific heat capacity of a solid heat sink may be greater than or equal to about 0.30 J/g·° C. By way of non-limiting example, copper has a heat capacity of about 0.38 J/g·° C.

In certain alternative variations, a thermally conductive component in heat transfer relationship with the light source to transfer heat to a heat sink involves employing a thermal energy storage or phase change material capable of absorbing heat. Thermal energy storage (TES) can capture and store heat via a chemical reaction, such as a hydration/dehydration reaction. Phase change materials can go through an endothermic reaction when heated, for example, from a solid to liquid phase transition to absorb heat. For example, either the LED light source or the thermal bridge in the antibacterial interior component may be in contact with or surrounded by the thermal energy storage or phase change material capable of absorbing heat. Examples of suitable phase change materials are hydrocarbons, organic molecules, fatty acids, and salt hydrates, which may have melting temperatures between −20 and 200° C. Additional phase change materials may be found in Applied Thermal Engineering, 23 (2003) pp. 251-283, the relevant portions of which are incorporated herein by reference in its entirety.

In certain aspects, like that shown in FIG. 5, the door panel (e.g., the first interior door panel component 114A and the second door panel component 114B) may be formed of at least one metal material, such as steel or aluminum alloys, by way of example. Thus, the thermal heat bridge 140 is physically attached via fasteners like bolts 142 to the LED light source(s) 120 and to a solid heat sink in the form of the door panel component, like the first interior door panel component 114A and the second door panel component 114B. The thermal bridge 140 may be in direct contact with adjacent components to transfer heat thereto. Thus, heat generated during operation of the LED light sources 120 is transferred away from the door handle component 108 and bezel 112 and into the door panel (e.g., the first interior door panel component 114A and the second door panel component 114B). In this manner, the region surrounding the LED light sources 120 within or adjacent to the door handle component 108 does not exceed a temperature of greater than about 100° C., optionally greater than about 90° C., optionally greater than about 80° C., optionally greater than about 70° C., and in certain variations, optionally greater than about 60° C., so as to prevent or minimize any damage.

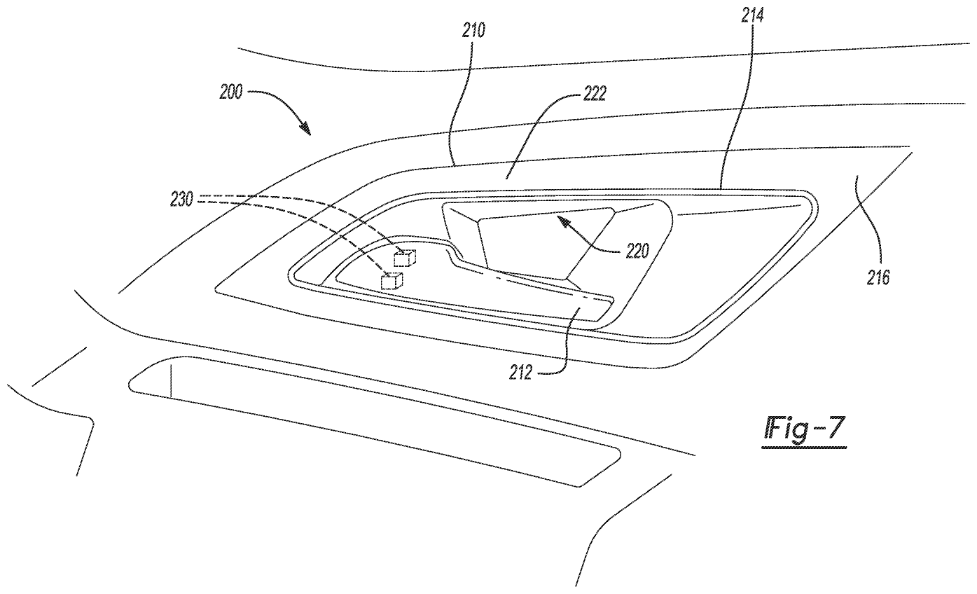

FIG. 6 shows an alternative variation of an antibacterial interior component 150 with another variation of a thermally conductive component. A door handle component 158 includes a door handle 160 disposed in a bezel 162 that may be integrated into a door panel (not shown). The handle 160 includes high touch surface regions 164 that are contacted by an occupant as they enter or exit the vehicle when the door is opened by the door handle 160. The bezel 162 defines a roof or upper portion 168 in which one or more high power LED light sources 170 are mounted. As will be appreciated by those of skill in the art, various other conventional components and connectors may be present that are not described and shown in FIG. 6. A thermally conductive component in the form of a plurality of heat exchange fins 180 is disposed over and in heat transfer relationship (e.g., direct contact) with the LED light source (s) 170. The plurality of heat exchange fins may include parallel fins that are spaced apart from one another and may form distinct units grouped together. The heat exchange fins 180 may be formed of a thermally conductive material having properties like those described above in the context of the thermal bridge 140 in FIG. 5. In one variation, the plurality of heat exchange fins 180 may be formed of a copper material. The LED light sources operate in a similar manner to those described above. When activated, the LED light sources 170 generate blue light (not shown) and heat that can be transferred to the plurality of heat exchange fins 180. The heat exchange fins 180 readily transfer heat away from the LED light sources 170 into the surrounding atmosphere, even if it is a static environment without air flow to maintain a temperature in the region of less than or equal to about 100° C. or the other maximum temperatures discussed above.

In yet other variations, the thermally conductive component in thermal communication with the LED light sources may include a potting compound to bridge the heat from the light source to the interior component. Potting compound may comprise a thermoset polymer, an epoxy, a urethane, or a siloxane polymer. The potting compound may further include thermally conductive material fillers that serve to increase thermal conductivity, such as, carbon/graphite/graphene, boron nitride (BN), aluminum nitride (AlN), metals, silicon nitride (Si₃N₄), alumina (Al₂O₃), magnesium oxide (MgO), and the like. In other variations, a thermally conductive material, such as a metal or graphite/graphene material or other thermally conductive material can be applied as a heat spreader on a back surface of the interior component to dissipate heat from the high energy light source.

FIGS. 7-9 show yet another variation of a self-sanitizing antibacterial interior component 200 for a vehicle in the form of a door handle component 210. A door handle 212 is disposed in a bezel 214 that is integrated into a door (an interior door panel 216) of a vehicle. The bezel 214 defines a confined space that includes a shadowed area or region 220 beneath an upper roof portion 222. In the variation in FIGS. 7-9, one or more high power LED light sources 230 are integrated into the door handle 212 itself. Thus, such an embodiment embeds one or more LEDs within a transparent handle, so that it is illuminated from the inside. The door handle 212 may be formed of a material that is transparent or transmissive to wavelengths of blue light generated by the high power LED light sources 230. For example, the door handle 212 may be formed of an acrylate. In one non-limiting example, the door handle may be formed of transparent polyamide, such as BASF ULTRAMID CLEAR™, which is stated to be approximately 82% transmissive in visible wavelengths up to a thickness of 1 mm and about 70% transmissive through a 2 mm thick material. In certain variations, one or more surfaces of the door handle 212 may have a roughened surface (in other words, a higher surface roughness) that serves to increase light scattering.

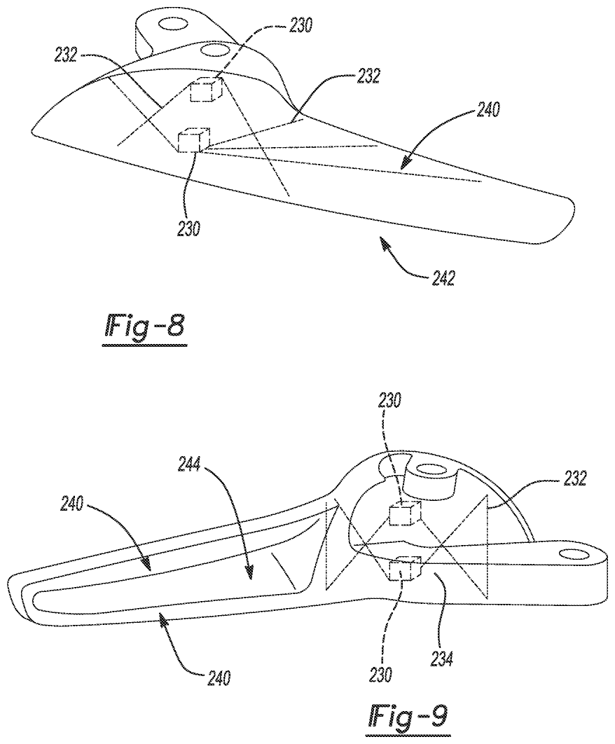

When activated, the LED light sources 230 generate blue light 232 as shown in FIGS. 8 and 9 that is directed from an interior 234 out towards a high touch surface 240 on door handle 212. FIG. 8 shows a front side 242 of the door handle 212 that faces the occupant in an occupant zone of the vehicle cabin, while FIG. 9 shows an interior or back side 244 of door handle 212 faces towards the bezel 214. The handle 212 includes the high touch surface regions 240 on both the front side 242 and back side 244, which are contacted by an occupant as they enter or exit the vehicle when the door is opened by the door handle 212. As shown in FIG. 1, these high touch surface regions 240 may be cast in a shadow in shadowed region 220 and are not illuminated. Further, at least some of these high touch surface regions 240 are not readily accessible for thorough cleaning by applying a cleanser or wiping. Thus, when the blue light 232 is generated by the high power LED light sources 230 of the antibacterial interior component 200, these high touch surface regions 240 can be treated to substantially kill bacteria.

The present disclosure further provides methods of operating an antibacterial interior component of a vehicle like those described above. In one variation, the method may include activating a light source comprising a light emitting diode (LED) in a first operational mode that generates blue light directed towards at least one touch surface in a confined space of the antibacterial interior component having a shadowed region. The blue light has a wavelength of greater than or equal to about 375 nm to less than or equal to about 425 nm. During this process, greater than or equal to about 90% of bacteria initially present on the at least one touch surface is killed. The method also includes transferring heat from the light source to a thermally conductive component either in or adjacent to the antibacterial interior component, so that a temperature in a region within or adjacent to the antibacterial interior component is less than or equal to about 60° C. In certain aspects, the method further comprises activating the light source comprising the light emitting diode (LED) in a second operational mode distinct from the first operational mode to illuminate the antibacterial interior component having a shadowed region. As discussed above, the first operational mode is used for killing bacteria and may have an optical power output of greater than or equal to about 0.5 W, while the second operational mode is used for illumination and may have an optical power output of less than or equal to about 0.5 W.

In certain aspects, an irradiance or flux density on the at least one touch surface during the first operational mode is greater than or equal to about 5 mW/cm², or any of the levels discussed previously above. Likewise, the light source may generate a radiant fluence on the at least one touch surface during the first operational mode of greater than or equal to about 5 J/cm² to less than or equal to about 50 J/cm² for bactericidal effect during the first operational mode.

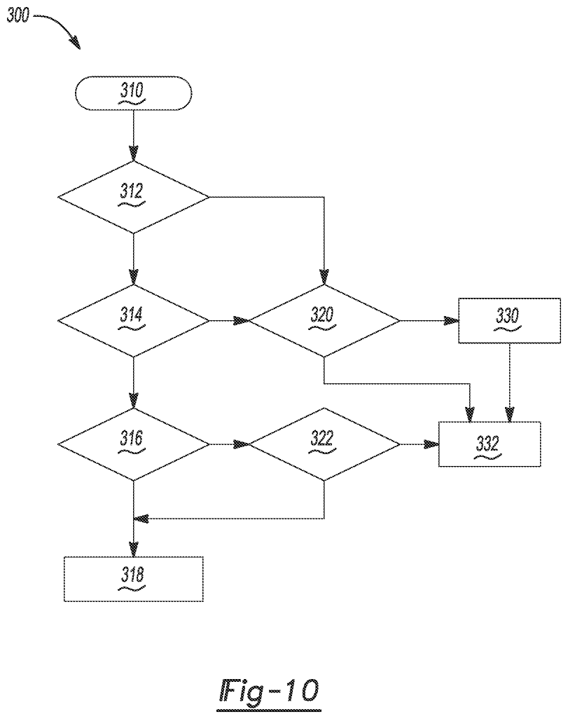

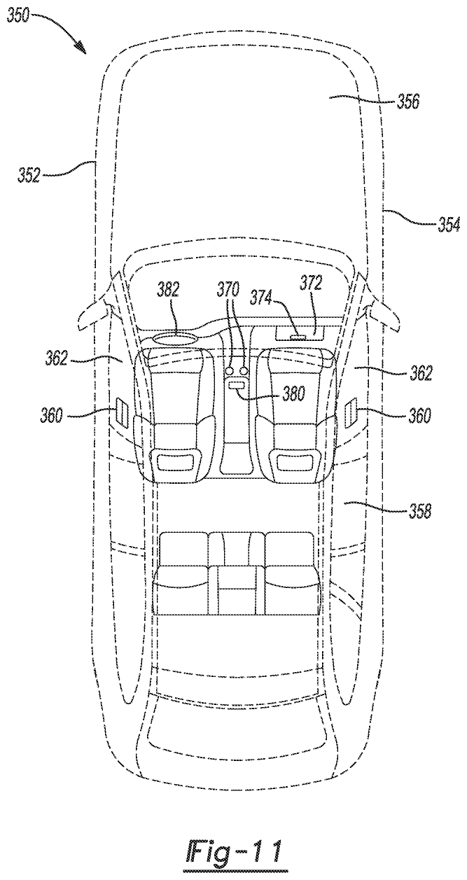

FIG. 10 is a schematic flowchart of a control algorithm for operation of a self-sanitizing interior component, which may be executed via a processor in the vehicle. Start 310 of the process leads to step 312 where it is determined whether it is dark outside the vehicle (e.g., by input from a sensor on the exterior of the vehicle). In one embodiment, such a sensor may be a light sensor for sensing a level of light in the exterior of the vehicle. Such input data may be communicated to the processor. If it is dark outside the vehicle at step 312, the method includes a step 314 of determining whether the vehicle is occupied, as will be described further below. If it is not dark outside as determined in step 312, the method progresses to step 320, where a sensor on the antibacterial interior component provides input as to whether the component has been used since the last sanitizing treatment (for example, indicating if the handle was pulled or other touch/capacitive contact has occurred).

If step 320 provides that the antibacterial interior component has been used, the process proceeds to step 330 where the high energy light source (LEDs) is activated in the first operational mode to emit high levels of blue light for a predetermined length of time corresponding to a timer for bactericidal effect. Such levels of high energy blue light for the first operational mode include those described previously above. In certain aspects, the predetermined length of time for bactericidal treatment with blue light may be greater than or equal to about 3 minutes to less than or equal to about 60 minutes, optionally greater than or equal to about 5 minutes to less than or equal to about 30 minutes. Thus, the processor may enable activation of the high energy light source to the first operational mode, if the cabin or interior of the vehicle is determined to be unoccupied based on input data from the sensor and other data. Based on the input data, the processor may activate or deactivate the high energy light source in the antibacterial interior component to a first operational mode. For example, if no motion within the interior or cabin of the vehicle is detected within a predetermined duration, for example for 5 minutes, then the processor may activate the high energy light source in the interior of the vehicle.

When the high energy light source is activated, it can generate and emit high energy blue light onto the high touch surfaces of the interior component thereby irradiating or reducing bacteria therefrom. The step 330 where the high energy light source (LEDs) is activated may have an automated or preselected density of energy (e.g., flux), time duration (e.g., via a timer), or any other suitable predetermined manner of ending step 330 to arrive at step 332 where the high energy light source is deactivated and the blue light is off. If the antibacterial interior component has not been used in step 320, the high energy light source (LEDs) are deactivated and the blue light is off at step 332.

With renewed reference to step 314, where it is determined whether the vehicle is occupied, if the sensor input indicates that there are occupants in the vehicle, the method progresses to step 316. One or more sensors may communicate data to the processor for determining the occupancy status in the vehicle interior/cabin. In one embodiment, such a sensor may be a motion sensor to sense movement or motion or a pressure sensor in the seat(s) within the interior of the vehicle. Such input data may be communicated to the processor. Step 316 ascertains whether the vehicle is stopped. This may be determined by inputting information from the battery charge state, speedometer, motion sensors, state of the engine or transmission operation (e.g., parked, in gear), and the like as input data communicated to the processor. If the vehicle is stopped, the method progresses to step 318, where the high energy light source (LEDs) are operated in a second operational mode at low power levels for illumination of the interior component (e.g., to illuminate the interior component for night visibility). Such power levels were discussed above. If the vehicle is determined to not be stopped at step 316, the method includes determining if the vehicle settings have been set so that running illumination is “on” at step 322. If the settings have running illumination set to on, the method progresses to step 318, where the high energy light source (LEDs) are operated in a second operational mode at low power levels for illumination of the interior component. If the settings have running illumination set to “off” at step 322, then the blue light is deactivated or off at step 332.

As discussed above, the processor in the vehicle may include at least one algorithm optionally, but not necessarily, having a plurality of steps or rules to activate/deactivate the high energy light source and further to operate it in either a first operational mode or a second operation mode within the interior or cabin of the vehicle. As input data is received from the sensors along with other data, the processor in the vehicle runs the control algorithm having a plurality of steps through which the processor undergoes to activate or inactivate the high energy light source. It is also to be understood that the algorithm steps discussed above may be modified or reduced as needed. Moreover, additional algorithm steps employing additional assessments and rules may be implemented.

In various aspects, the present disclosure thus provides a self-sanitizing antibacterial interior component and methods for operating such a self-sanitizing antibacterial component in a vehicle. The interior components incorporate blue light for bacterial reduction and are more effective in treating the entire component to kill bacteria, including shadowed regions and do so with faster treatment times and lower overall electrical power requirements. Moreover, the self-sanitizing antibacterial interior component have a thermally conductive component, for example, a thermal bridge that provides heat sinking to avoid overheating and potential damage to the surrounding area. The high power light sources for generating blue light are integrated into the interior components to illuminate surfaces shadowed by general interior lighting. The integrated lighting has at least two intensity levels to provide night time illumination, as well as a bacterial kill mode.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An antibacterial interior component in a vehicle comprising:
at least one touch surface in a confined space having a shadowed region;
a light source comprising a light emitting diode (LED) that generates light having a wavelength of greater than or equal to about 375 nm to less than or equal to about 425 nm directed towards the at least one touch surface for killing bacteria; and
a thermally conductive component in heat transfer relationship with the light source to transfer heat to a heat sink either in or adjacent to the antibacterial interior component.

2. The antibacterial interior component of claim 1, wherein the light source comprises a light emitting diode (LED) having an optical power output capacity of greater than or equal to about 1 W.

3. The antibacterial interior component of claim 1, wherein the light source is configured to have a first operational mode for killing bacteria on the at least one touch surface having an optical power output of greater than or equal to about 0.5 W and a second operational mode for illumination having an optical power output of less than or equal to about 0.5 W.

4. The antibacterial interior component of claim 3, wherein the first operational mode is configured for killing greater than or equal to about 90% of bacteria initially present on the at least one touch surface.

5. The antibacterial interior component of claim 1, wherein the light source generates a radiant fluence on the at least one touch surface of greater than or equal to about 5 J/cm² to less than or equal to about 50 J/cm².

6. The antibacterial interior component of claim 1, wherein an irradiance on the at least one touch surface is greater than or equal to about 1 mW/cm².

7. The antibacterial interior component of claim 1, wherein the antibacterial interior component is selected from the group consisting of: a door handle, a cup holder, a glove compartment, a latch, a handle, a steering wheel, and combinations thereof.

8. The antibacterial interior component of claim 1, wherein a region surrounding the light source does not exceed a temperature of greater than about 100° C.

9. The antibacterial interior component of claim 1, wherein the thermally conductive component has a thermal conductivity of greater than or equal to about 20 Wm/K.

10. The antibacterial interior component of claim 1, wherein the thermally conductive component is a thermal heat bridge attached to the light source and to a solid heat sink.

11. The antibacterial interior component of claim 1, wherein antibacterial interior component is a door handle, the light source is disposed in or near a bezel that surrounds the door handle, and the heat sink is a door panel.

12. The antibacterial interior component of claim 1, wherein the thermally conductive component is a thermal bridge connecting the light source to a solid component in a door panel or the thermally conductive component comprises a plurality of heat exchange fins.

13. The antibacterial interior component of claim 1, wherein the thermally conductive component comprises a phase change material that is configured to undergo an endothermic reaction to absorb heat.

14. The antibacterial interior component of claim 1, wherein the thermally conductive component is a potting compound or a heat spreader disposed on a surface of the antibacterial interior component.

15. The antibacterial interior component of claim 1, wherein antibacterial interior component is a door handle and the light source is disposed in the door handle.

16. An antibacterial door handle component in a vehicle comprising:
at least one touch surface on a door handle in a door handle component in a door panel, the door handle component comprising a confined space having a shadowed region;
a light source comprising a light emitting diode (LED) associated with the door handle component that generates light having a wavelength of greater than or equal to about 375 nm to less than or equal to about 425 nm directed towards the at least one touch surface on the door handle for killing greater than or equal to about 90% of bacteria present on the at least one touch surface; and
a thermally conductive component in heat transfer relationship with the light source to transfer any heat generated to the door panel.

17. A method of operating an antibacterial interior component of a vehicle, the method comprising:
activating a light source comprising a light emitting diode (LED) in a first operational mode that generates blue light having a wavelength of greater than or equal to about 375 nm to less than or equal to about 425 nm directed towards at least one touch surface in a confined space of the antibacterial interior component having a shadowed region, so that greater than or equal to about 90% of bacteria initially present on the at least one touch surface is killed; and
transferring heat from the light source to a thermally conductive component either in or adjacent to the antibacterial interior component, so that a temperature in a region within or adjacent to the antibacterial interior component is less than or equal to about 100° C. during the activating.

18. The method of claim 17, further comprising activating the light source comprising the light emitting diode (LED) in a second operational mode distinct from the first operational mode to illuminate the antibacterial interior component having a shadowed region, wherein the first operational mode for killing bacteria has an optical power output of greater than or equal to about 0.5 W and the second operational mode for illumination has an optical power output of less than or equal to about 0.5 W.

19. The method of claim 17, wherein an irradiance on the at least one touch surface during the first operational mode is greater than or equal to about 10 mW/cm².

20. The method of claim 17, wherein the light source generates a radiant fluence on the at least one touch surface during the first operational mode of greater than or equal to about 5 J/cm² to less than or equal to about 50 J/cm².
